(19) United States
(12) Patent Application Publication
(10) Pub. No.: US 2005/0049709 A1
(43) Pub. Date: Mar. 3, 2005

(54) Glenoid component of a shoulder prosthesis and complete shoulder prosthesis incorporating such a component

(76) Inventor: Alain Tornier, Saint Ismier (FR)

Correspondence Address: Ralph A. Dowell of DOWELL & DOWELL P.C., 2111 Eisenhower Ave., Suite 406, Alexandria, VA, 22314, US

(21) Appl. No.: 10/925,142

(22) Filed: Aug. 25, 2004

(30) Foreign Application Priority Data
Aug. 25, 2003 (FR) 03 10141

(51) Int. Cl. A61F 2/40

(52) U.S. Cl. 623/19.13

(57) ABSTRACT

The glenoid component according to the invention comprises a metal body of which the inner face is adapted to be immobilized on the glenoid cavity of a shoulder and of which the outer face bears a concave articulating surface adapted to cooperate with a humeral component. This articulating surface extends on the periphery by a convex surface forming, at least in part, the edge of the body.

### FIELD OF THE INVENTION

The present invention relates to a glenoid component of a shoulder prosthesis, as well as to a complete shoulder prosthesis comprising such a component.

### BACKGROUND OF THE INVENTION

In the domain of shoulder prostheses, it is current to use a glenoid component comprising a concave body made of polyethylene on which bears a convex head, made of metal, of a humeral component. This is, for example, the case in U.S. Pat. No. 5,593,448 and U.S. Pat. No. 6,406,495. In fact, it is largely admitted that it is the humeral head which is the piece under most mechanical stress during the functioning of the prosthesis.

However, this widely employed solution presents a certain number of drawbacks associated with the wear of the polyethylene glenoid body. In effect, it is observed that this polyethylene body wears out fairly rapidly, all the more SO as the contact between the polyethylene body and the harder humeral head is formed over an area smaller for the polyethylene body than for the humeral head. Moreover, in the event of poor positioning of the glenoid body with respect to the humeral head, the wear is poorly distributed. Under the effect of efforts of tipping generated by the poorly positioned humeral head, the prosthesis in that case risks being deteriorated either by unsealing of the glenoid component or by dislocation of the joint.

In order to overcome these drawbacks, it was proposed in the past either to increase the thickness of the polyethylene body, which, however, offsets the surface of articulation with the humeral head from its anatomical position, or to interpose between the polyethylene body and the osseous glenoid cavity, a metallic insert (called “metal-back” glenoid component), as described in U.S. Pat. No. 4,550,450. However, this latter solution remains expensive and currently leads to a disconnection of the polyethylene body with respect to the metal insert, especially when the positioning between the humeral head and the glenoid body is not anatomically satisfactory.

It is an object of the present invention to propose a glenoid component which both limits the risks of deterioration and unsealing of this component and which efficiently cooperates with a humeral prosthetic component, while being economical and easy to implant.

### SUMMARY OF THE INVENTION

To that end, the invention relates to a glenoid component of a shoulder prosthesis, comprising a metal body adapted, on one side, to be immobilized on the glenoid cavity of a shoulder and bearing, on the opposite side, a concave articulating surface adapted to cooperate with a humeral component of the prosthesis, which articulating surface extends on the periphery by a convex surface forming, at least in part, the edge of the body.

By using the metal body of the glenoid component according to the invention, the rigidity of this component is significantly increased, without necessitating a great thickness. There is virtually zero wear of this metal piece, thus considerably reducing the risks of its deterioration. In functioning, the curvature of the articulating surface is provided to limit the risks of dislocation of the humeral component and the blunt periphery of this surface avoids marking, or indenting, the humeral component. In addition, compared to the “metal-back” glenoid components, the component according to the invention is more economical and easier to implant.

Other characteristics of this glenoid component, taken separately or in all technically possible combinations, are set forth in the dependent claims 2 to 12.

The invention also relates to a complete shoulder prosthesis which comprises a glenoid component as defined hereinabove and a humeral component bearing a convex surface for articular cooperation with the concave surface of the glenoid component. Such a prosthesis presents the advantage that the wear is borne on the humeral component, i.e. over a greater surface borne by a thicker material. In addition, when, after several years, it is necessary to change the expendable parts of the prosthesis subjected to wear, only the head of the humeral component is to be changed, rendering the corresponding operation more rapid and easier.

According to an advantageous characteristic of this complete shoulder prosthesis, the convex surface of the humeral component is borne by a part of said component constituted by a material less rigid than the metal of the body of the glenoid component, particularly a plastics material.

### BRIEF DESCRIPTION OF THE DRAWINGS

The invention will be more readily understood and other advantages thereof will appear more clearly in the light of the following description, given solely by way of example and made with reference to the accompanying drawings, in which:

FIG. 1 schematically shows a prosthesis according to the invention, implanted on a patient.

FIG. 2 is a view in elevation of the glenoid component of the prosthesis of FIG. 1.

FIG. 3 is a view taken in the direction of arrow III indicated in FIG. 2.

FIG. 4 is a view in perspective of a variant glenoid component according to the invention.

FIG. 5 is a section along plane V of the component of FIG. 4, implanted on the glenoid cavity of a patient.

FIGS. 6 and. 7 are views in perspective, from different angles, of another variant glenoid component according to the invention, and

FIG. 8 is a section along a substantially median plane of the component of FIG. 6 and 7, implanted on the glenoid cavity of a patient.

### DESCRIPTION OF PREFERRED EMBODIMENTS

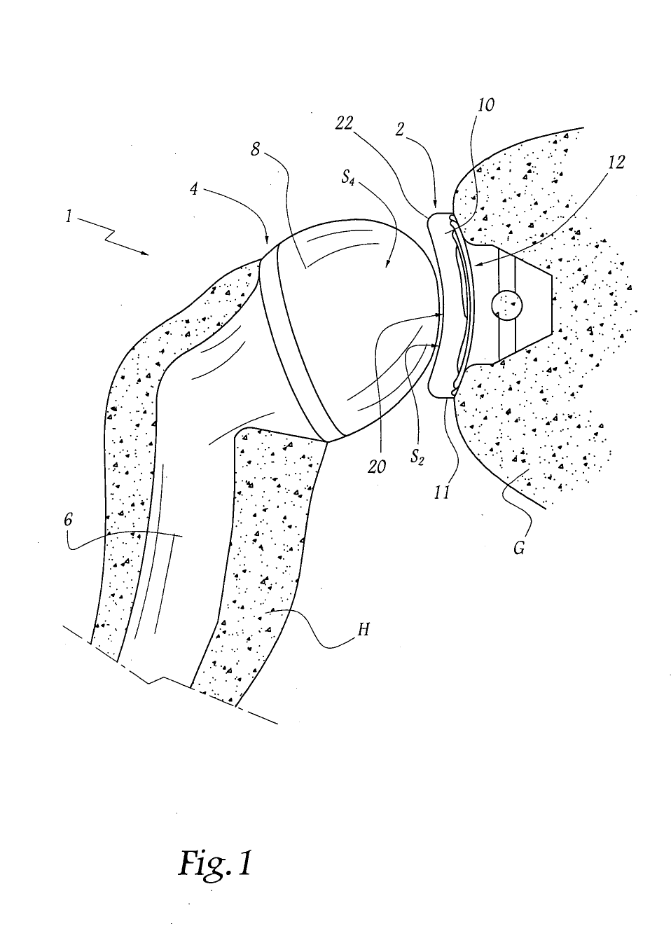

Referring now to the drawings, the prosthesis 1 shown in FIG. 1 comprises a first component 2 fixed on the glenoid cavity G of a shoulder, as well as a second component 4 fixed in the corresponding humerus H.

The component 4 comprises, on the one hand, a stem 6 intended to be anchored in the medullary cavity of the humerus H, and, on the other hand, a hemispherical head 8 defining a convex surface S4 in the form of a frustum of sphere.

The stem 6 is made of metal, while the head 8 is, at least in its peripheral part, made of less rigid material, particularly polyethylene, in particular high density polyethylene (HDPE). The head 8 is fixedly connected on the stem 6 by any known means, for example by interlocking. The head 8 is advantageously adaptable on existing humeral stems.

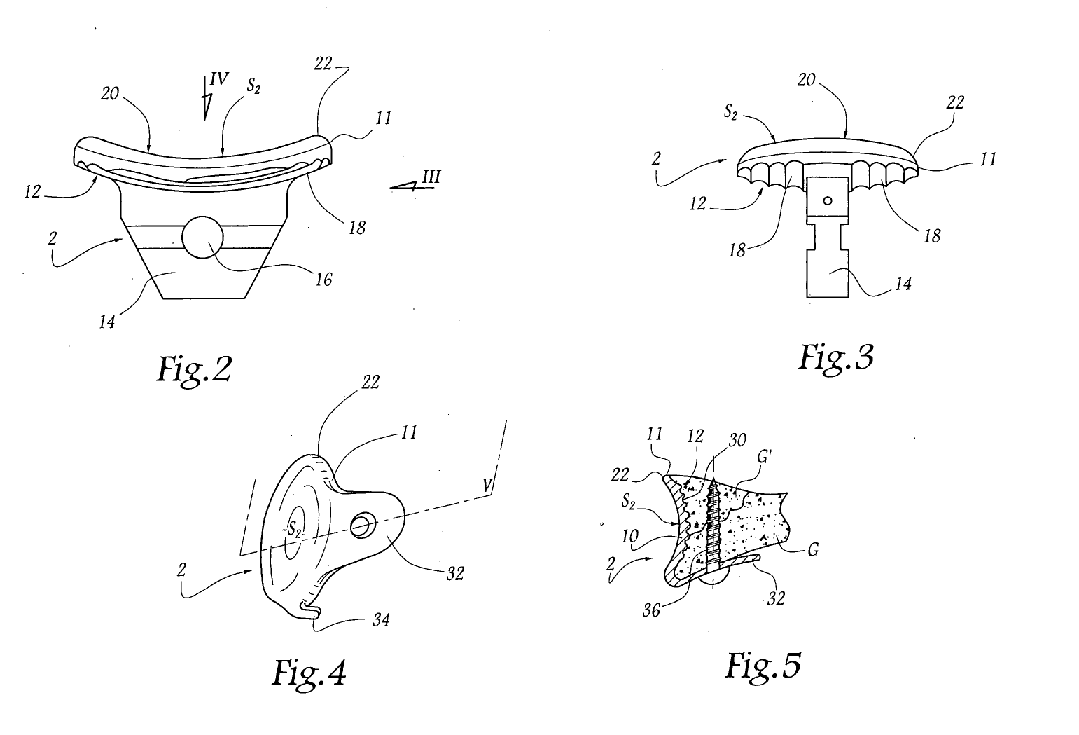

For convenience, the following description, particularly that in connection with the glenoid component 2, will be made, considering that this component is in its position of implantation with respect to the glenoid cavity G. More precisely, considering the component 2 shown in FIGS. 2 and 4, the terms “lower” and “upper” respectively designate the left- and right-hand parts of FIGS. 2 and 4, the terms “outer” and “inner” designate the upper and lower parts of FIG. 2, and the terms “anterior” and “posterior” respectively designate the upper and lower parts of FIG. 4.

The component 2 of FIGS. 2 to 4 comprises a metal body 10 in one piece, presenting, in the antero-posterior and lower/upper directions, dimensions which are clearly greater than its thickness in the inner/outer direction. The peripheral edge of the body 10 is referenced 11.

The body 10 presents an inner face 12 adapted to be immobilized on the glenoid cavity G. More precisely, this face 12 is provided with a projecting wing 14 intended to be deeply anchored in the osseous glenoid cavity and in which a hole 16 is made, inside which a screw for immobilization may be inserted. The wing 14 is advantageously metallic and integral with the body 10.

On either side of the wing 14, the face 12 defines concave surfaces 18 for abutment on the glenoid cavity of the patient's scapula. These surfaces advantageously bear grooves which extend in the lower/upper direction and which are intended to come into engagement with the osseous wall of the glenoid cavity G.

The inner face 12 is advantageously coated at least in part by a layer of calcium hydroxyapatite intended to facilitate osteo-integration once the glenoid component 2 has been implanted.

The body 12 also presents an outer face 20 bearing a concave surface S₂ adapted to cooperate in articular manner with the convex surface S₄ of the humeral head 8. In manner known per se, the respective geometries of these surfaces S₂ and S₄ are conceived in order to reproduce the anatomical articular behaviours of the shoulder as faithfully as possible. Moreover, the curvature of the surface S₂ is adapted to limit as much as possible the risks of dislocation of the humeral head 8 when the prosthesis 1 is subjected to current stresses.

The concave surface S₂ extends on its periphery by a convex surface 22 which forms the start of the edge 11 on the outer side of the glenoid component. In other words, the inner part of the edge 11, substantially planar in the embodiment of FIGS. 1 to 3, and the surface S₂ of the outer face 20 are continuously joined by the incurved surface 22. The blunt shape of this surface 22 limits the risks of cut, indenting or marking of the head 8 when the prosthesis 1 is under stress. By way of example, the minimum radius of curvature of the surface 22 is of the order of 1 to 3 mm for a glenoid component intended for an adult.

In order to facilitate obtaining of the blunt surface 22, the latter preferably joins the articulating surface S₂ tangentially, thus also avoiding any risk of indenting the humeral head 8.

When the complete prosthesis 1 is implanted and under stress, the humeral head 8 abuts on the outer face 20 of the glenoid body 10, the surfaces S₂ and S₄ being articulated on each other.

Compared to the prostheses of the prior art in which the wear is essentially supported by a glenoid component made of polyethylene, the prosthesis according to the invention presents the advantage of transferring such wear on the humeral head 8, i.e. over the surface S₄ whose area of contact is greater than that of surface S₂. The corresponding wear is slowed down. Moreover, the thickness of polyethylene bearing the surface S₄ may be provided to be greater than that envisaged for a glenoid component of the prior art, the useful volume available at the level of the humeral head being greater than that generally available for the glenoid component, unless a prosthesis is formed whose behaviour would be very far from the anatomical behaviour. The life of the prosthesis 1 is thus increased.

When the prosthesis 1 has attained its predetermined life duration, the expendable part formed by the humeral head 8 is changed, which is easier and more rapid than changing the glenoid component as in the present-day shoulder prostheses.

In addition, the glenoid body 10 being made of metal, it is considerably rigid and is more easily anchored in the glenoid cavity G. In addition to the connection means described hereinabove, the body 10 is, in a variant embodiment (not shown), pierced with through holes opening out on the articulating surface S₂ and adapted to receive screws for anchoring in the bone, particularly self-blocking screws. With a present-day glenoid component of which the body is made of a less rigid material, such as polyethylene, the use of such self-blocking screws cannot be envisaged. Thus, insofar as the glenoid component according to the invention allows a more efficient osseous fixation, its positioning can be envisaged with or without cement.

FIGS. 4 and 5 show a variant of the glenoid component 2 which essentially differs from the embodiment of the preceding Figures at the level of its inner side. In effect, the inner face 12, not presenting a projecting wing, is intended to be cemented on the glenoid cavity G. To that end, the face 12 bears in its central part a surface 30 adapted to facilitate setting of the cement. This surface is for example shot-peened, sanded or polished.

In order to fix the component 2 solidly to the glenoid cavity, the body 10 is provided with an anterior tab 32 and with a lower catch 34. These elements 32 and 34 are in the form of metal tongues, integral with the body 10, which project from the body towards the glenoid cavity and which are joined to the body at the level of its edge 11. More precisely, the tab 32 and the catch 34 tangentially join corresponding peripheral parts of the blunt surface 22.

The tab 32 extends sufficiently along the glenoid bone G to receive a screw 36 for anchoring in the bone, shown in FIG. 5. This screw is preferably self-blocking with respect to the tab and advantageously extends at a angle of retro-version allowing an osseous reconstruction of the glenoid cavity by implantation of a catch G′ in the posterior part of the bone.

As for the catch 34, it makes it possible to form a lower bearing for the glenoid cavity G.

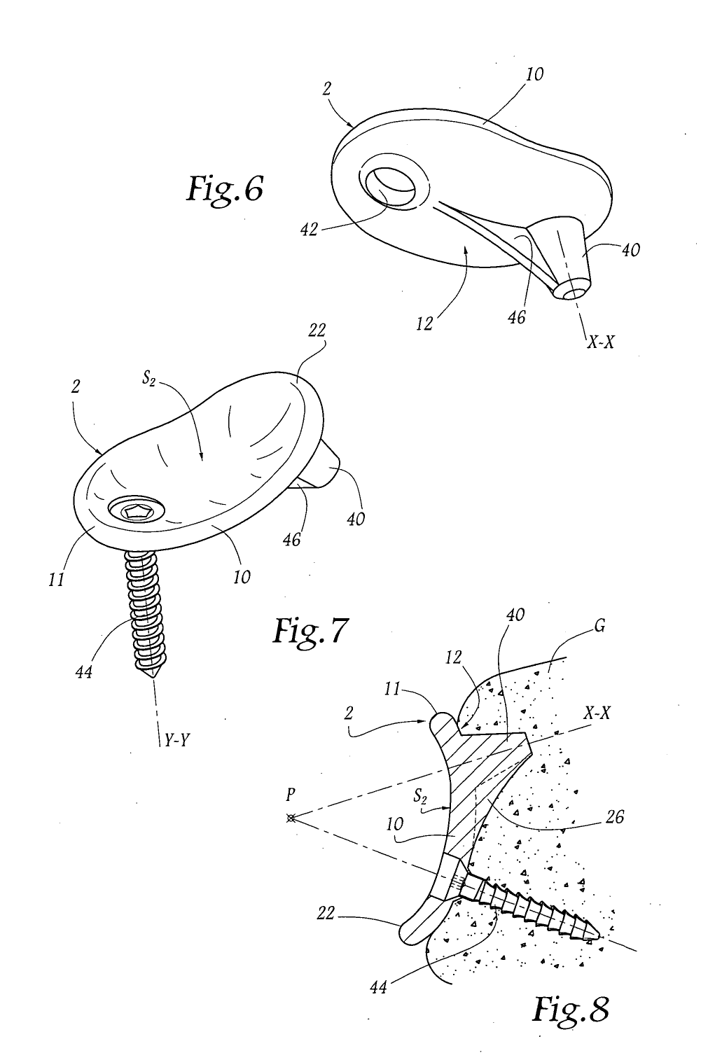

FIGS. 6 to 8 show another variant of the glenoid component 2 which essentially differs from the embodiment of FIGS. 1 to 3 at the level of its inner side. In effect, the projecting wing 14 is replaced by a stud 40 for anchoring in the bone of the glenoid cavity G. This stud is of substantially truncated form, of axis X-X, which projects from the inner face 12 of the body 10, being integral with the body. The surface of the stud 40 is divergent in the direction of the face 12 so as to facilitate introduction thereof in the bone, which was possibly previously hollowed out in substantially corresponding manner.

The stud 40 extends from the upper part of the inner face 12 while the lower part presents a through bore 42 adapted to receive a screw 44 for anchoring in the bone in the lower part of the glenoid cavity G, this screw being similar to screw36 of FIG. 5. This screw 44 is preferably self-blocking with respect to the bore 42.

When the screw 44 is received in the bore 42, it extends in length about a central axis Y-Y which defines, with axis X-X, a substantially vertical plane corresponding to the plane of FIG. 8. In this plane, axes X-X and Y-Y are inclined with respect to each other and intersect at a point P located on the outer side of the body 10, i.e. towards the centre of curvature of the surface S₂. In other words, the axes X-X and Y-Y diverge from each other on the inner side of the body 10, this ensuring a reliable anchoring, both the upper and lower parts of the glenoid cavity G being under stress.

In order to limit the risks of the component 2 tipping around a substantially vertical direction, a rib 46 is integral with the face 12 of the body 10 and extends in the plane of FIG. 8, from the stud 40 up to the periphery of the bore 42, with a continuously decreasing thickness.

According to a variant embodiment (not shown), the stud 40 may be internally bored along its axis X-X, so as to be able to receive another screw for anchoring in the bone.

Other variants of and modifications to the glenoid component and the complete prosthesis described hereinabove may, in addition, be envisaged. In particular, the means for connection with the glenoid cavity or the humerus envisaged hereinabove are in no way limiting and may be modified by the person skilled in the art. Similarly, the bearing surface(s) 18 or 30 of the glenoid body 10 may be substantially planar like the glenoid component of FIGS. 6 to 8, depending on the osseous state of the glenoid cavity and the geometry of the resection effected for positioning the component 2. The glenoid cavity 2 may be composed of a plurality of metal parts connected to one another.

Furthermore, insofar as the glenoid body 10 is made of metal, it is easy to provide for this body to extend as far as the acromial part of the patient's scapula, so as to increase the connection of the glenoid component on the bone of the shoulder.

1. Glenoid component of a shoulder prosthesis, comprising a metal body adapted, on one side, to be immobilized on the glenoid cavity of a shoulder and bearing, on the opposite side, a concave articulating surface adapted to cooperate with a humeral component of the prosthesis, which articulating surface extends on the periphery by a convex surface forming, at least in part, the edge of the body.

2. The component of claim 1, wherein said convex surface and the articulating surface join each other tangentially.

3. The component of claim 1, wherein the minimum radius of curvature of said convex surface is included between about 1 and 3 mm.

4. The component of claim 1, wherein the metal body is in one piece.

5. The component of claim 1, wherein the metal body is provided with at least one through orifice opening out on the articulating surface and adapted to receive means for anchoring the component in the bone of the shoulder, particularly self-blocking anchoring means.

6. The component of claim 1, wherein the metal body is provided, on the side intended to be immobilized on the glenoid cavity, with at least two elements for anchoring in the bone, defining respective directions of application in the bone of the glenoid cavity, which are divergent with respect to each other on moving away from the metal body.

7. The component of claim 1, wherein the metal body is provided, on the side intended to be immobilized on the glenoid cavity, with at least one projecting tongue which extends in line with a part of said convex surface.

8. The component of claim 7, wherein the tongue(s) is/are integral with the body.

9. The component of claim 7, wherein the or one of the tongues forms, preferably in the anterior part of the glenoid component, a tab for fixation in the bone, adapted to receive a screw for anchoring in the bone.

10. The component of claim 7, wherein the or one of the tongues forms, preferably in the lower part of the glenoid component, a catch for bearing on the glenoid cavity.

11. The component of claim 1, wherein the side of the metal body adapted to be immobilized on the glenoid cavity is covered with an active layer in order to facilitate osteo-integration, particularly with a layer of calcium hydroxyapatite.

12. The component of claim 1, wherein the side of the metal body adapted to be immobilized on the glenoid cavity presents a surface state provided to facilitate the cementing of the glenoid component.

13. Complete shoulder prosthesis, wherein it comprises a glenoid component according to claim 1, and a humeral component bearing a convex surface for articular cooperation with the concave surface of the glenoid component.

14. The prosthesis of claim 13, wherein the convex surface of the humeral component is borne by a part of said component constituted by a material less rigid than the metal of the body of the glenoid component, particularly a plastics material.
